SEQUENCE LISTING (6 sequences)

SEQ ID NO: 1 - DNA, 21 bp, Artificial Sequence
  Primer used for VCAM-1 sense
ctcaatggggtggtaaggaat

SEQ ID NO: 2 - DNA, 22 bp, Artificial Sequence
  Primer used for VCAM-1 antisense
gggggcaacgttgacataaaga

SEQ ID NO: 3 - DNA, 23 bp, Artificial Sequence
  Primer used for VLA-4 sense
tgtctgccagggtgtgagtccat

SEQ ID NO: 4 - DNA, 24 bp, Artificial Sequence
  Primer used for VLA-4 antisense
agcaccaccgagtagccaaacagc

SEQ ID NO: 5 - DNA, 24 bp, Artificial Sequence
  Primer used for identification of RAGE transgene
agcggctggaattgaaactgaaca

SEQ ID NO: 6 - DNA, 22 bp, Artificial Sequence
  Primer used for identification of RAGE transgene
gaaggggcaagggcacaccatc

(19) United States
(12) Patent Application Publication
(10) Pub. No.: US 2007/0167360 A1
(43) Pub. Date: Jul. 19, 2007

(54) Methods for treating multiple sclerosis

(76) Inventors: Shi Du Yan, Tenafly, NJ (US); David Stern, Cincinnati, OH (US)

Correspondence Address: COOPER & DUNHAM, LLP, 1185 AVENUE OF THE AMERICAS, NEW YORK, NY, 10036, US

(21) Appl. No.: 10/577,506

(22) PCT Filed: Oct. 28, 2004
(86) PCT No.: PCT/US04/36170

§ 371 Date: Nov. 30, 2006

Related U.S. Application Data
(60) Provisional application No. 60/516,328, filed on Oct. 31, 2003.

(51) Int. Cl. A61K 38/17

(52) U.S. Cl. 514/12

(57) ABSTRACT

This invention provides a method for treating a subject afflicted with multiple sclerosis comprising administering to the subject a therapeutically effective amount of soluble receptor for advanced glycation endproducts (sRAGE). This invention further provides a method for inhibiting CD4⁺ T-cell migration comprising contacting the CD4⁺ T-cell with soluble receptor for advanced glycation endproducts (sRAGE). This invention further provides a method for inhibiting chemokine receptor activation in a subject comprising administering to the subject a therapeutically effective amount of soluble receptor for advanced glycation endproducts (sRAGE).

This application claims priority of U.S. Provisional Application No. 60/516,328, filed on Oct. 31, 2003, the contents of which are hereby incorporated by reference. This invention was made with support under United States

Throughout the application, various publications are referenced. Full citations for these publications may be found immediately preceding the claims. The disclosures of these publications are hereby incorporated by reference into this application in order to more fully describe the state of the art as of the date of the invention described and claimed herein.

Government Grant Nos. NS42855, AI44927 and AI46132 from the United States Public Health Service. Accordingly, the United States Government has certain rights in the subject invention.

### BACKGROUND OF THE INVENTION

Multiple sclerosis (MS), the most frequently encountered autoimmune disease of the central nervous system (CNS), results from inhibition of nerve conduction due to destruction of myelin sheaths by immune/inflammatory mechanisms (1). Although the precise events triggering MS in man have not precisely been defined, the presence of T-lymphocytes reactive with components of myelin sheaths, such as myelin basic protein, myelin oligodendrocyte glycoprotein and proteolipid protein, are thought to have prominent roles (2). For example, CD4+ T-cells with similar immunoreactivity are encephalitogenic in animal models (3, 4).

Receptor for Advanced Glycation Endproduct (RAGE) is a member of the immunoglobulin superfamily of cell surface molecules first discovered because of its interaction with products of nonenzymatic glycoxidation termed Advanced Glycation Endproducts (AGEs) (5). Subsequently, two endogenous ligands of RAGE have been identified, members of the S100/calgranulin family and the high mobility group I-type polypeptide amphoterin (6, 7). Whereas amphoterin appears to be expressed at high levels in tumors and during development (7-9), S100/calgranulins in the extracellular space are well-known for their association with inflammatory disorders; they have been found in colitis, arthritis, cystic fibrosis, and chronic bronchitis (10). RAGE has been identified as a central signal transduction receptor mediating effects of S100/calgranulins on key cellular targets, including mononuclear phagocytes (MPs), lymphocytes and vascular endothelium (6). The potential physiologic significance of this interaction was emphasized by inhibition of the delayed-type hypersensitivity response by blockade of RAGE-S100/calgranulin interaction (6).

### SUMMARY OF THE INVENTION

This invention provides a method for treating a subject afflicted with multiple sclerosis comprising administering to the subject a therapeutically effective amount of soluble receptor for advanced glycation endproducts (sRAGE).

This invention further provides a method for inhibiting CD4⁺ T-cell migration comprising contacting the CD4⁺ T-cell with soluble receptor for advanced glycation endproducts (sRAGE).

This invention further provides a method for inhibiting chemokine receptor activation in a subject comprising administering to the subject a therapeutically effective amount of soluble receptor for advanced glycation endproducts (sRAGE).

This invention further provides an article of manufacture comprising (a) a packaging material having therein soluble receptor for advanced glycation endproducts (sRAGE) and (b) instructions for using the sRAGE in treating multiple sclerosis.

This invention further provides an article of manufacture comprising (a) a packaging material having therein soluble receptor for advanced glycation endproducts (sRAGE) and (b) instructions for using the sRAGE in inhibiting CD4⁺ T-cell migration in a subject.

Finally, this invention provides an article of manufacture comprising (a) a packaging material having therein soluble receptor for advanced glycation endproducts (sRAGE) and (b) instructions for using the sRAGE to inhibit CD4⁺ T-cell migration in a subject.

### BRIEF DESCRIPTION OF THE FIGURES

FIG. 1

Immunolocalization of RAGE and S100 antigens in the spinal cord of MS patients (A-D), mice induced to develop EAE (E), and naive controls. Spinal cord sections from patients with MS (A1, B1, C1-2) or age-matched controls (A2, B2, C3) were stained with H&E (A), a-RAGE IgG (B) or a-S100b IgG (C). Panels A3, B3 and C4 show image analysis of representative sections from 5 patients with MS and 3 controls displaying area occupied by the indicated stained profile (A3, H&E-stained nuclei; B3, a-RAGE IgG; C4, a-S100b IgG). In panels B-C, the substrate is aminoethylcarbazole. Panel D displays sequential double-staining of MS spinal cord tissue: D1-2, a-RAGE IgG and a-mouse macrophage IgG (a-Mf), respectively; D3-4, a-RAGE IgG and a-CD4 IgG, respectively. In panels D1&3, the substrate is aminoethylcarbazole. In panels D2&4, the substrate is diaminobenzidine. Spinal cord sections from mice induced to develop EAE (E) double-stained with either a-RAGE IgG (E1) and rat a-mouse macrophage (F4/80; E2) or a-RAGE IgG (E3) and a-CD4 IgG (E4). In panels E1&3, the substrate is fast red, and in panels E2&4, the substrate is aminoethylcarbazole. Arrowheads denote cells costaining for RAGE and the indicated marker in D3,4 & E. Scale bar indicates: A1-2, 50 μm; B1-2, C1-3, 20 μm; D1-2, 5 μm; D3-4, 10 μm; E1-4, 5 μm.

FIG. 2

Effect of RAGE blockade on EAE induced by 1-9NAc MBP. A. Mice (B10.PL) were immunized with 1-9NAc MBP and received pertussis toxin. Treatment with sRAGE (50 μg/day, IP) or vehicle (phosphate-buffered saline; IP) was begun at the time of MBP-peptide immunization and continued until day 35. Symptoms were scored as described. B. Induction of EAE was performed as above, and animals were treated with the indicated concentration of sRAGE (once daily, IP) ox vehicle alone (0, IP). Clinical score was determined on day 35. C. Representative H&E stained spinal cord sections display the extent of cellular infiltration in mice induced to develop EAE treated with either vehicle (C2) or sRAGE (C3) compared with naive animals (C1). C4 displays image analysis of area occupied by nuclei from samples similar to that in (C1-3) from three mice in each of the groups. Marker bar indicates 5 μm. D. Immunoblotting of spinal cord protein extracts (100 μg/lane) for RAGE, S100b and β-actin antigens was performed as described. Samples are from: 1-3, naive animals; 4-5, EAE-induced and treated with vehicle; and, 6-8, EAE-induced and treated with sRAGE (50 μg/day; IP). E. Immunoprecipitation of splenic protein extracts from mice induced to develop EAE treated with either vehicle or sRAGE (50 μg/day; IP) for 21 days. At the time of sample harvest, vehicle-treated animals showed level 4 symptoms Immunoprecipitation (samples were 200 μg total protein, in each case) employed a-S100b IgG and immunoblotting used a-RAGE IgG (as described in the text). Samples were from mice induced to develop EAE treated with either sRAGE (lane 1) or vehicle (lane 2), or from naive mice (lane 3). In lane 4, the sample was from EAE-induced animals treated with sRAGE, and a-S100b IgG was replaced with nonimmune IgG. Migration of simultaneously run molecular weight standards is shown on the far right in kD in D-E. Results shown are representative of at least three repetitions.

FIG. 3

Mechanisms of sRAGE-mediated suppression of EAE induced by 1-9NAc MBP. A. 3H-thymidine incorporation by splenocytes (A1) or lymph node cells (A2) from 1-9NAc MBP-treated animals treated with either sRAGE (50 μg/day; IP) or vehicle (phosphate buffered saline; IP) on day 21 (at time corresponding to level 4 symptoms in the vehicle-treated group). Results from naive animals are shown as a control (N=4/group in each case). B. Gel shift analysis was performed with 32P-labelled consensus NF-kBii probe and nuclear extracts from spleens (10 μg/sample) of animals immunized with 1-9NAc MBP and treated with either vehicle (lane 2; IP) or sRAGE (lanes 3-4; 50 μg/day; IP). Results in splenic nuclear extracts from naive mice are shown in lane 1. Nuclear extracts from EAE-induced mice receiving vehicle (as in lane 2) were also incubated with 32P-labelled NF-kB probe in the presence of an 100-fold excess of unlabelled probe (lane 5). C-D. Ribonuclease protection assays with probes for the indicated chemokines (C) or chemokine receptors (D1 & D2) employed RNA harvested from spinal cords of mice immunized with 1-9NAc MBP followed by treatment with vehicle (lanes 4-6) or sRAGE (lanes 7-9). Samples were obtained at the time of peak symptoms in the vehicle-treated group. Samples were also obtained from naive mice (lanes 1-3). Densitometric analysis of the data is shown to the right of the gel (* indicates p<0.01). E. RT-PCR analysis of cDNA prepared from spinal cord RNA of mice of the above experimental groups using primers for VCAM-1, VLA-4 (a4), or β-actin. F. Zymogram to assess MMP 9 activity in spinal cord extracts of the above three groups of mice. Densitometric analysis of the data is shown above the zymogram. Experimental methods are described in the text. Results shown are representative of at least three repetitions.

FIG. 4

Activation of 1AE10 cells and adoptive transfer of EAE. A. Immunoblotting of activated 1AE10 cells using a-RAGE IgG, a-S100b IgG and anti-β-actin IgG. 1AE10 cells were activated for the indicated times with 1-9NAc MBP (5 μg/ml), and protein extracts (100 μg/lane) were prepared for reduced SDS-PAGE/immunoblotting. Densitometric analysis of the data is shown. B. 1AE10 cells were activated with 1-9NAc MBP as above, and, on day 4, total RNA was harvested. RT-PCR was performed with primers for murine VLA-4 (a4) and β-actin: lane 1, untreated 1AE10 cells (no 1-9 NAc MBP and no antibody fragments); lane 2, 1AE10 cells+1-9 NAc MBP (5 μg/ml); lane 3, 1AE10 cells+1-9 NAc MBP peptide+nonimmune F(ab′)₂ (1 μg/ml); lane 4, 1AE10 cells+1-9 NAc MBP peptide+a-RAGE F(ab′)₂ (1 μg/ml). C. 1AE10 cells were activated in vitro for 4 days (as above) and then adoptively transferred into prepared B10.PL mice. Mice were treated with either rabbit a-RAGE F(ab′)₂, rabbit nonimmune F(ab′)₂ (NI; 50 μg/day in each case; IP) or phosphate-buffered saline (IP) for 17 days, and symptoms were scored for up to 35 days. D. H&E stained sections of spinal cord from the experiment in D: D1, naive; D2, EAE-induced and treated with phosphate-buffered saline; D3, EAE-induced and treated with a-RAGE F(ab′)₂ (a-RAGE); and, D4, EAE-induced and treated with nonimmune F(ab′)₂ (NI). D5 displays image analysis in which area occupied by nuclei in the H&E stained sections is shown (this analysis utilized 3 mice in each experimental group). Scale bar indicates 5 μm. E. Fluorescently-labelled, activated 1AE10 cells were adoptively transferred into prepared B10.PL mice. Animals received either PBS (E1) anti-RAGE F(ab′)₂ (a-RAGE; E2) or nonimmune F(ab′)₂ (NI; 20 μg/day; IP; E3), and were sacrificed on day 3. Scale bar indicates 5 μm. *P<0.001. E4 displays the number of fluorescently-labelled cells per high power field when experiments with 3 mice per group were analyzed. F. 51Cr-labelled, activated 1AE10 cells were adoptively transferred into B10.PL mice using the same protocol as in E. Spinal cord (F1), spleen (F2) and liver (F3) were harvested after 24 hours and radioactivity was determined (N=4 in each experimental group). G. Chemotaxis of 1AE10 cells. Cells were added to the upper compartment of microchemotaxis chambers and the chemotactic stimulus, S100b (G1), was added to the lower compartment. Where indicated, the chemotactic stimulus was also added to the upper compartment. In certain experiments, 1AE10 cells were preincubated with anti-RAGE F(ab′)₂ (a-RAGE) or nonimmune F(ab′)₂ (NI; 1 μg/ml) for 2 hr at 37° C., and then they were added to the upper compartment of the microchemotaxis chamber (G2). In G3, FMLP was also used as the chemotactic stimulus in the presence of a-RAGE or NI. H. Zymograms of whole cell lysates from 1-9NAc MBP-activated 1AE10 cells. Cells were incubated with anti-RAGE F(ab′)₂ (a-RAGE; 5 μg/ml) or nonimmune F(ab′)₂ (NI; 5 μg/ml) during exposure to MBP peptide. Lane 1, untreated 1AE10 cells (no 1-9 NAc MBP and no antibody fragments); lane 2, 1AE10 cells+1-9 NAc MBP (5 μg/ml); lane 3, 1AE10 cells+1-9 NAc MBP peptide+anti-RAGE F(ab′)₂ (1 μg/ml); lane 4, 1AE10 cells+1-9 NAc MBP peptide+nonimmune F(ab′)₂ (1 μg/ml). Results shown are representative of at least three repetitions.

FIG. 5

Effect of sRAGE on spontaneous EAE in T/a-β- mice. A. T/a-β- mice were treated with either sRAGE (50 μg/day; IP) or vehicle (PBS; IP) from days 21 to day 65. Symptoms were scored as described. B. H&E-stained spinal cord sections from T/a-β- mice treated with sRAGE (B1) or vehicle (B2). Marker bar indicates 5 μm. B3 shows image analysis of the area occupied by nuclei in sections such as that shown in B1-2 (3 animals/group for this analysis). Results shown are representative of at least three repetitions.

FIG. 6

Effect of RAGE on 1-9NAc MBP-induced EAE: studies in transgenic mice with targeted overexpression of the receptor in CD4+ T-cells and mononuclear phagocytes. A. PCR analysis for the CD4-DN-RAGE transgene to identify genotypes in a representative litter (+, Tg CD4-DN-RAGE; −, nontransgenic control [nonTg]). B. Immunoblotting of CD4+ T-cells isolated from spleens of Tg CD4-DN-RAGE or Tg CD4− wt (wild-type)RAGE mice (10% reduced SDS-PAGE; 100 μg total protein/lane). Samples for the lanes were from: 1-2, Tg CD4-DN-RAGE; 3, nonTg littermate; and, 4, Tg CD4-wtRAGE. Migration of simultaneously run molecular weight standards is shown on the right. C. Chemotaxis of CD4+ T-cells isolated from spleens of Tg CD4-wtRAGE and CD4-DN-RAGE mice. Cells were added to the upper compartment of the microchemotaxis chamber, and S100b (indicated concentration) was added to the lower compartment. D. Tg CD4-DN-RAGE mice and nonTg controls were immunized with 1-9NAc MBP/pertussis toxin and symptoms were scored over 40 days. E. H&E staining of spinal cord sections from the experiment shown in panel D (E1, untreated naive mouse (nonTg) littermate); E2, nonTg littermate induced to develop EAE (as above); and E3, Tg CD4-DN-RAGE induced to develop EAE). E4 shows image analysis of area occupied by nuclei in sections from mice in each of the groups (N=3/group). Note that the degree of cellular infiltration in Tg CD4-DN-RAGE induced to develop EAE was the same as in naive Tg CD4-DN-RAGE animals (not shown). Marker bar indicates 5 μm. F. Tg MSR-DN-RAGE mice and nonTg controls were immunized with 1-9NAc MBP/pertussis toxin and symptoms were scored on day 35 (N is greater than or equal to 4/group). Results shown are representative of at least three repetitions.

### DETAILED DESCRIPTION OF THE INVENTION

Terms

“Activity” of a protein shall mean any enzymatic or binding function performed by that protein.

“Administering” an agent can be effected or performed using any of the various methods and delivery systems known to those skilled in the art. The administering can be performed, for example, intravenously, orally, nasally, via the cerebrospinal fluid, via implant, transmucosally, transdermally, intramuscularly, and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering agents. (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

“RAGE” shall mean, without limitation, receptor for advanced glycation endproducts, and can be from human or any other species which produces this protein. The nucleotide and protein (amino acid) sequences for RAGE (both human and murine and bovine) are known. The following references, inter alia, provide these sequences: Schmidt et al, J. Biol. Chem., 267:14987-97, 1992; and Neeper et al, J. Biol. Chem., 267:14998-15004, 1992. Additional RAGE sequences (DNA sequences and translations) are available from GenBank.

“Equivalent”, when used in relation to a specified daily dosage, shall mean that when a dose of sRAGE is administered to the subject at a frequency other than every day, that dose, if administered daily, would fall within the specified daily dosage. For example, a 150 mg dose of sRAGE administered once every 10 days is equivalent to a 15 mg dose of sRAGE administered daily.

“Subject” shall mean any animal, such as a human, non-human primate, mouse, rat, guinea pig or rabbit.

“Treating” a disorder shall mean slowing, stopping or reversing the disorder's progression. In the preferred embodiment, treating a disorder means reversing the disorder's progression, ideally to the point of eliminating the disorder itself.

### EMBODIMENTS OF THE INVENTION

This invention provides a method for treating a subject afflicted with multiple sclerosis comprising administering to the subject a therapeutically effective amount of soluble receptor for advanced glycation endproducts (sRAGE). In the preferred embodiment, the subject is human.

In one embodiment of the instant method, the therapeutically effective amount of sRAGE is an amount between about 150 μg sRAGE/kg of subject/day and 15 mg sRAGE/kg of subject/day, or its equivalent. In another embodiment of the instant method, the therapeutically effective amount of sRAGE is an amount between about 500 μg sRAGE/kg of subject/day and 5 mg sRAGE/kg of subject/day, or its equivalent. In another embodiment of the instant method, the therapeutically effective amount of sRAGE is about 1.5 mg sRAGE/kg of subject/day, or its equivalent.

This invention further provides a method for inhibiting CD4⁺ T-cell migration comprising contacting the CD4⁺ T-cell with soluble receptor for advanced glycation endproducts (sRAGE). In the preferred embodiment, the CD4⁺ T-cell is a human CD4⁺ T-cell.

In one embodiment of the instant method, the CD4⁺ T-cell is present in a subject, and the contacting with sRAGE is performed by administering a therapeutic amount of sRAGE to the subject. In the preferred embodiment, the subject is human.

In one embodiment of the instant method, the therapeutically effective amount of sRAGE is an amount between about 150 μg sRAGE/kg of subject/day and 15 mg sRAGE/kg of subject/day, or its equivalent. In another embodiment of the instant method, the therapeutically effective amount of sRAGE is an amount between about 500 μg sRAGE/kg of subject/day and 5 mg sRAGE/kg of subject/day, or its equivalent. In another embodiment of the instant method, the therapeutically effective amount of sRAGE is about 1.5 mg sRAGE/kg of subject/day, or its equivalent.

This invention further provides a method for inhibiting chemokine receptor activation in a subject comprising administering to the subject a therapeutically effective amount of soluble receptor for advanced glycation endproducts (sRAGE). In the preferred embodiment, the subject is human.

In one embodiment of the instant method, the chemokine receptor is selected from the group consisting of CCR1, CCR2, CCR5, CXCR2, CXCR4, VCAM-1, VLA-4, MMPS receptor, RANTES receptor, MIP-1β receptor, MIP-1a receptor, MIP-2 receptor, JE/MCP-1-receptor and TCA-3 receptor.

In a further embodiment of the instant method, the therapeutically effective amount of sRAGE is an amount between about 150 μg sRAGE/kg of subject/day and 15 mg sRAGE/kg of subject/day, or its equivalent. In another embodiment of the instant method, the therapeutically effective amount of sRAGE is an amount between about 500 μg sRAGE/kg of subject/day and 5 mg sRAGE/kg of subject/day, or its equivalent. In another embodiment of the instant method, the therapeutically effective amount of sRAGE is about 1.5 mg sRAGE/kg of subject/day, or its equivalent.

This invention further provides an article of manufacture comprising (a) a packaging material having therein soluble receptor for advanced glycation endproducts (sRAGE) and (b) instructions for using the sRAGE in treating multiple sclerosis.

This invention further provides an article of manufacture comprising (a) a packaging material having therein soluble receptor for advanced glycation endproducts (sRAGE) and (b) instructions for using the sRAGE in inhibiting CD4⁺ T-cell migration in a subject.

Finally, this invention provides an article of manufacture comprising (a) a packaging material having therein soluble receptor for advanced glycation endproducts (sRAGE) and (b) instructions for using the sRAGE to inhibit CD4⁺ T-cell migration in a subject.

This invention is illustrated in the Experimental Details section which follows. This section is set forth to aid in an understanding of the invention but is not intended to, and should not be construed to limit in any way the invention as set forth in the claims which follow thereafter.

### EXPERIMENTAL DETAILS

Introduction

These experiments show upregulation of RAGE and its S100/calgranulin ligands in affected spinal cord from patients with MS and mice subject to experimental autoimmune encephalitis (EAE). Models of EAE provide experimental systems to analyze molecular mechanisms underlying EAE, including models of disease elicited in genetically susceptible strains of mice by immunization with components of myelin sheaths (11, 12), adoptive transfer of encephalitogenic T-cell clones (3, 13), and spontaneous disease in T-cell receptor transgenic mice (11). These models afford an opportunity to examine how RAGE impacted on an important inflammatory paradigm. An unique profile of RAGE inhibition of the evolving immune/inflammatory response emerged; blockade of RAGE suppressed activation of MBP-specific T-cells with respect to their ability to infiltrate the CNS. The predominant effect of RAGE was localized to CD4+ T-cell compartment, as studies with transgenic mice expressing a dominant-negative RAGE transgene targeted to CD4+ T-cells were resistant to myelin basic protein (MBP)-induced EAE. These studies highlight a new facet of the biology of EAE/MS, RAGE-ligand interaction, and suggest that inhibition of RAGE could provide a means of protecting animals from disease even when primed encephalitogenic T-cells are present during the course of an acute exacerbation.

Materials and Methods

I. RAGE-Related Reagents and RAGE Transgenic Mice.

Murine soluble (s) RAGE was expressed using the baculovirus system and purified to homogeneity as described previously (16). Rabbit anti-murine RAGE IgG was prepared and characterized as described (6), and nonimmune rabbit IgG was similarly processed. For in vivo studies, F(ab′)₂ fragments were prepared from rabbit IgG's using a commercially available kit (Pierce, Rockfold, Ill.). The latter materials (sRAGE, anti-RAGE and nonimmune IgG/F(ab′)₂) were devoid of contaminating endotoxin based on the limulus amebocyte assay (Sigma) at a protein concentration of 5 mg/ml.

Transgenic Mice

Generation and characterization of transgenics (Tg), Tg CD4-DN-RAGE and MSR-DN-RAGE mice, will be described in detail elsewhere. Briefly, for Tg MSR-DN-RAGE mice, the macrophage scavenger receptor promoter/enhancer (57) was used to drive expression of the dominant-negative (DN) human RAGE cDNA. DN-RAGE refers to a tail-deleted variant of RAGE which has properties of a dominant-negative receptor with respect to RAGE-mediated cellular activation (6). For Tg CD4-DN-RAGE mice, expression of the human DN-RAGE cDNA was driven by regulatory elements in the CD4 locus (the promoter, proximal/distal enhancers and a silencer) in a 992 bp construct (generously provided by Dr. Gerald Siu, Columbia) previously used to make Tg mice in which expression of reporter genes is directed to mature CD4+ T-cells (38, 39). The DN-RAGE (1.1 kb) construct was subcloned into the CD4 Tg vector, and transgenic casettes (4656 bp) were created by releasing the SacI-XhoI fragments from the CD4-DN-RAGE construct (7.5 kb). Transgenic cassettes were microinjected into mouse oocytes of the B10.PL (for CD4-DN-RAGE) or B6CBAF1/J (Tg MSR-DN-RAGE) strains. After matings with males, founders were identified by Southern blotting, and transmission of the transgene was verified. Tg MSR-DN-RAGE mice were then backcrossed five times into the B10.PL background (controls for these experiments were nonTg littermates). Tg CD4-RAGE mice were also prepared as described above using full-length RAGE.

II. Induction of EAE and RAGE blockade.

MBP-immunization. EAE was induced in B10.PL mice (4-6 months of age; female) by subcutaneous immunization of 1-9NAc MBP (Ac-Ala-Ser-Gln-Lys-Arg-Pro-Ser-Gln-Arg; made in the Peptide Core Laboratory of Columbia; 0.1 mg/animal) emulsified in complete Freund's adjuvant (58). Pertussis toxin (0.1 μg/mouse) was injected intravenously 24 and 72 hrs later. The protocol for blockade of RAGE was to treat animals with either sRAGE (IP) or vehicle alone (IP), after immunization with 1-9Nac MBP and injection of pertussis toxin were completed (after the first 72 hrs), for at least an additional 21 days (because of this extended time interval, foreign antibody to RAGE could not be administered without inciting an immune response). Animals were evaluated for clinical symptoms and spinal cord pathology. Clinical symptoms were monitored daily according to the following scoring system (3): 0, no signs; 1, weakness of the tail, 2, mild paresis of hind limbs (paraparesis); 3, severe paraparesis; 4, complete paralysis of hind limbs (paraplegia) or the limbs of one side (hemiplegia); and 5, death.

Administration of Encephalitogenic CD4 Th1 T cells (1AE10) (32)

An encepholitogenic Th1 clone (1AE10) was generated as described. To establish an EAE model using this clone, B10.PL mice were sublethally irradiated (350 R) and after 24 hours were injected intravenously with 8-10×106 cells. The 1AE10 cells were activated by 1-9NAc MBP for 4 days prior to injection. Pertussis toxin was also injected at 0.1 μg/mouse as above. Animals were monitored daily and EAE was evaluated. 1AE10 effectively induced the clinical syndrome of EAE in B10PL mice with 100% efficiency. Where indicated, activated 1AE10 cells were fluorescently labelled using the Vybrant carboxyfluorescein diacetate succinimidyl ester cell tracer kit (V-12883; Molecular probes) and adoptively transferred into B10.PL mice prepared as above. 51Cr-labelling of 1AE10 cells was accomplished as described (59). T/a-β- mice, prepared and characterized as described (36), were treated with sRAGE (50 μg/day; IP) from age 20-21 days to age 60-65 days.

III. Histology/Immunohistology

Formalin-fixed paraffin embedded autoptic MS spinal cord tissue was obtained from the Department of Pathology (Columbia, N.Y.; N=5 for MS and N=3 for age-matched control). Sections were cut (5-6 μm) and immunostaining was performed with rabbit anti-RAGE IgG (as above; 50 μg/ml), murine monoclonal anti-CD68 IgG (20 μg/ml; Dako), murine monoclonal anti-CD4 IgG (5 μg/ml; Sigma), and rabbit antisera to S100b (1:100 dilution; Sigma). Sites of primary antibody binding were visualized with secondary antibodies using the Biotin ExtrAvidin kit (Sigma) using the manufacturer's instructions. Mouse tissue was processed as above and the following primary antibodies were employed: rabbit anti-RAGE IgG (50 μg/ml; as above), rat anti-mouse CD4 IgG (10 μg/ml; Pharmingen), and rat anti-mouse F4/80 IgG (5 μg/ml; Pharmingen). Semiquantitation of inflammatory infiltrates was determined by evaluation of the area occupied by nuclei in H&E stained sections per high power field (5 fields per slide) using the Universal Imaging System. Similar image analysis was used to quantify RAGE-positive cells in EAE-induced mice.

IV. Characterization of the Effects of RAGE blockade.

T-Cell Proliferation

Single cell suspensions, prepared from draining lymph nodes and spleens (5×105 cells per well in each case) were plated in flat bottom 96-well plates in serum free HL-1 media (BioWittacker) supplemented with L-glutamine at 1 mM. 1-9NAc MBP was added (0.1-80 μM). During the last 18 hours of the 4 day culture period, 3H-thymidine was added (1 μCi/well) and incorporation of radiolabel was measured by liquid scintillation counting.

Assessment of Th Phenotype of 1-9NAc MBP-Specific CD4+ T Cells

EAE was induced by immunizing B10.PL mice with 1-9NAc MBP and sRAGE treatment was performed as described above. Lymph-node and splenic cells from EAE-induced mice were assayed immediately for their Th phenotype by cytoplasmic cytokine staining. Briefly, lymph node and splenic cells were stimulated in vitro by exposure to phorbol ester (0.02 μg/ml) and Ionomycin (0.4 μM/ml) for 1 hour. Then, brefeldin A was added for an additional 4 hours to block the secretion of cytokines. The cells were permeabilized, and stained for intracellular INF-g and IL-5 using a kit (Pharmingen, San Diego, Calif.) and analyzed by FACS. In experiments for assaying Th phenotype of 1-9NAc MBP reactive T cells, animals were immunized with 1-9NAc MBP (0.1 mg/animal; SQ) in Complete Freund's Adjuvant (CFA) in the presence or absence of sRAGE treatment. Seven days later, lymphocytes from draining lymph nodes and spleens were harvested from those animals and their Th phenotypes were assayed.

Immunoblotting RAGE and S100

Spinal cords were harvested from mice, homogenized in tris-buffered saline (pH 7.4) with protease inhibitors (PMSF, 100 μg/ml; aprotonin, 1 μg/ml), tissue debris was removed by low speed centrifugation and the supernatant was boiled in reducing SDS gel sample buffer. Electrophoresis (10% for RAGE and 15% for S100) was performed under reducing conditions followed by transfer of proteins to nitrocellulose, blocking of membranes with nonfat dry milk (5%) and incubation with primary antibodies (murine monoclonal anti-RAGE IgG, 10 μg/ml, and rabbit anti-S100b IgG, 10 μg/ml). Sites of primary antibody binding were identified the ECL method (Amersham) as described by the manufacturer. As a control for protein loading and degradation, immunoblotting also employed murine monoclonal anti-β-actin IgG (100 ng/ml; Sigma). For analysis of CD4+ T cells from spleens of Tg CD4-wtRAGE and Tg CD4-DN-RAGE mice, the population of CD4+ T cells was isolated using Dynal beads for mouse CD4 according to the manufacturer's protocol (Dynal Biotech).

Immunoprecipitation was performed on protein extracts of spleen (tissue was homogenized was processed as above for immunoblotting) using rabbit anti-S100b IgG (100 μg/ml; Dako), followed by addition of protein A/G linked to agarose (Pierce) and washing with phosphate-buffered saline (pH 7.4) containing Tween 20 (0.05%). Agarose was then boiled in reduced SDS gel sample buffer and reducing SDS-PAGE (15%) was performed, followed by transfer of proteins to nitrocellulose and immunoblotting with mouse anti-RAGE IgG (3 μg/ml) as above.

Gel shift analysis was performed on nuclear extracts of spinal cord using 32P-labelled and unlabelled consensus NF-kB probes, as described previously (60).

Zymograms to assess the activity of MMP9 and 2 were performed using homogenates of spinal cord and extracts of 1AE10 cells according to the manufacturers' instructions (Invitrogen).

Ribonuclease Protection Assays/RT-PCR

Spinal cord RNA was harvested using Trizol (InvitroGen) and total RNA was labelled with 32P and used for ribonuclease protection assays with a Riboquant kit (for chemokine receptors, mCR-5, mCR-6; for chemokines, mCR-5, Pharmingen). Reverse transcription was done with Superscript II (Life Technologies), primed by oligo-dT following the manufacturer's protocol. For cDNA from spinal cord or 1AE10 cells, thermocycling parameters were: 94° C., 2 min, 1 cycle; 94° C. for 30 sec, 56° C. for 45 sec, 72° C. for 1.0 min, 38 cycles; 72° C. for 5 min, 1 cycle). The following primers were used: for VCAM-1 sense (5′-CTCAATGGGGTGGTAAGGAAT-3′) (SEQ. ID. NO: 1) and antisense (5′-GGGGGCAACGTTGACATAAAGA-3′) (SEQ. ID. NO: 2); for VLA-4 (integrin a4) sense (5′-TGTCTGCCAGGGTGTGAGTCCAT-3′) (SEQ. ID. NO: 3) and antisense (5′-AGCACCACCGAGTAGCCAAACAGC-3′) (SEQ. ID. NO: 4); and, for mouse β-actin primers were from Clontech. The expected size of amplicons was 470 bp (for VCAM-1) and 581 bp (for VLA-4). The following primers were used for identification of the RAGE transgene (spanning from 146 bp to 847 bp): sense (5′-AGCGGCTGGAATTGAAACTGAACA-3′) (SEQ. ID. NO: 5) and antisense (5′-GAAGGGGCAAGGGCACACCATC-3′) (SEQ. ID. NO: 6). These primers detected an amplicon of ≈700 bp when Tg CD4-wtRAGE mice were studied and ≈600 bp when Tg CD4-DN-RAGE mice were analyzed (because of truncation of the cytosolic tail of RAGE in the DN-RAGE construct. Thermocycling parameters for the latter experiments were: 95° C. for 3 min, 1 cycle; 95° C. for 20 sec, 57° C. for 30 sec, 72° C. for 1 sec, 35 cycles; and, 72° C. for 7 min, 1 cycle.

V. In Vitro Studies with 1AE10 Cells

Conditions for Activation

1AE10 cells were activated in the presence of 1-9NAc MBP (10 μg/ml) for 4 days in the presence of anti-RAGE F(ab′)₂ or nonimmune F(ab′)₂.

Cell migration assays CD4+ T cells were prepared from spleen, and migration assays were performed using 48-well microchemotaxis chambers (Neuro-Probe, Bethesda, Md.) containing a polycarbonate membrane with 8.0 μm pores (Neuro-Probe). For cell migration studies, suspensions of T cells (2×104 cells/well) were added to the upper compartment of the microchemotaxis chamber, and chemotactic agents were placed in the lower and/or upper compartments (S100b; Calbiochem, LaJolla, Calif.). Chambers were incubated for 45 min at 37° C. in an humidified carbon dioxide (5%)/air atmosphere. Cells attached to the upper side of the filter were removed manually by scraping, and cells migrating through pores of the membrane and emerging on the lower side were visualized by staining with Coomassie blue (Sigma). The latter cells were counted under the light microscope (400× magnification). For each filter, the number of cells in three adjacent fields was counted and the average was determined. Migrating T cells were quantified from at least 4 separate filters and the results were averaged. The latter average was considered to represent the number of cells migrating across the filter under a particular condition. The migration assay was repeated twice with different cell preparations with consistent results. FMLP (10-6 M; Sigma) was used as a positive control for cell migration studies.

### RESULTS

I. Expression of RAGE and S100 Proteins in MS and Murine EAE.

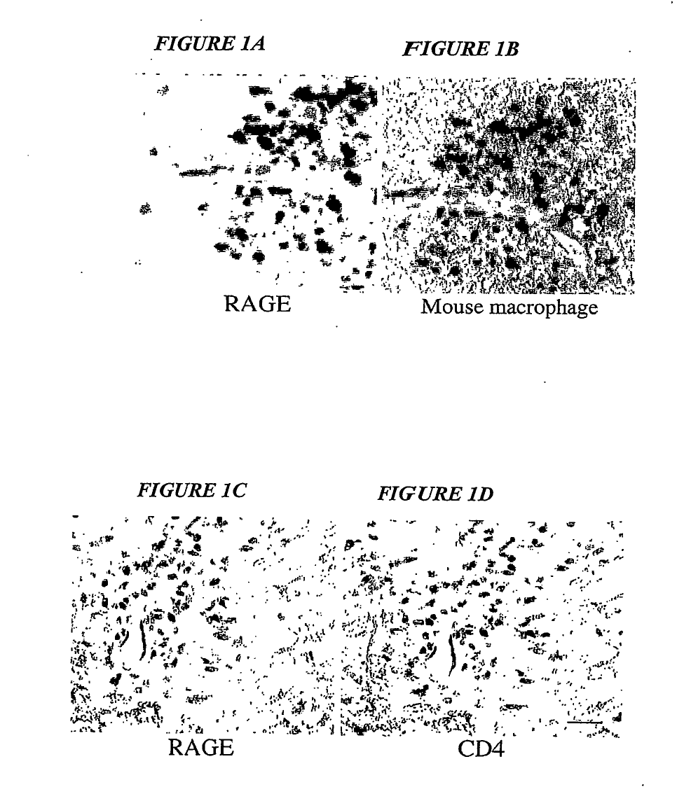

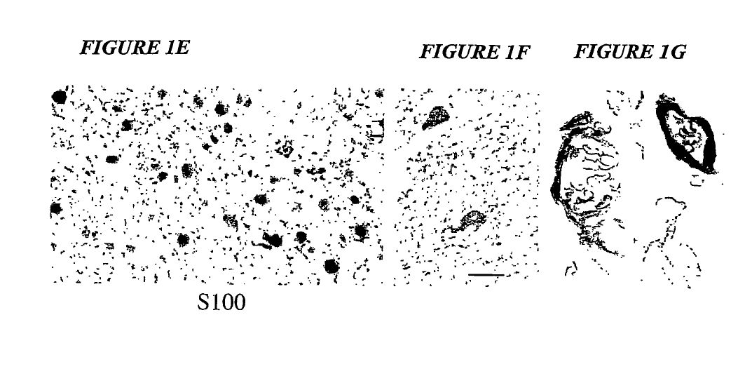

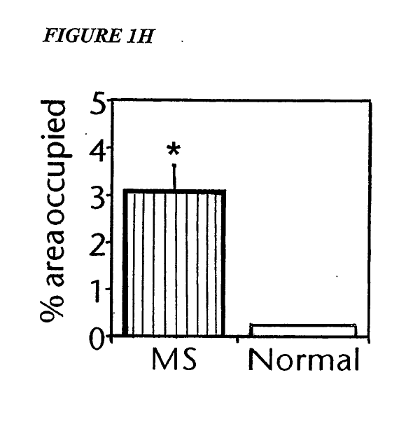

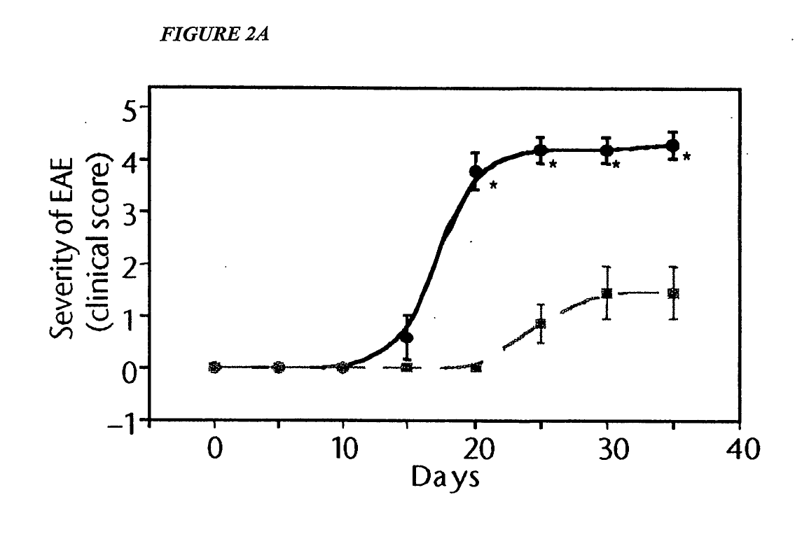

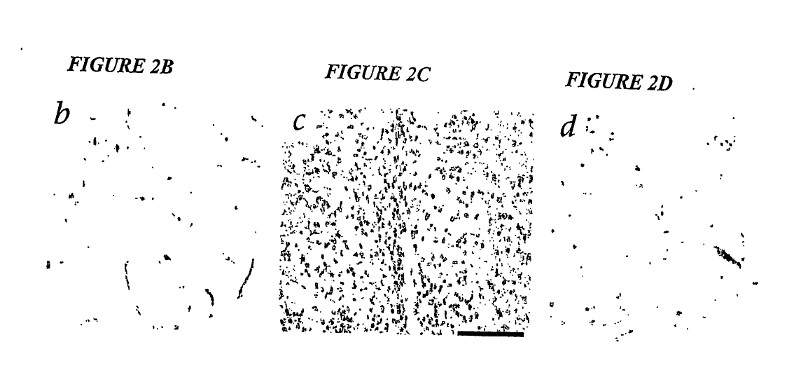

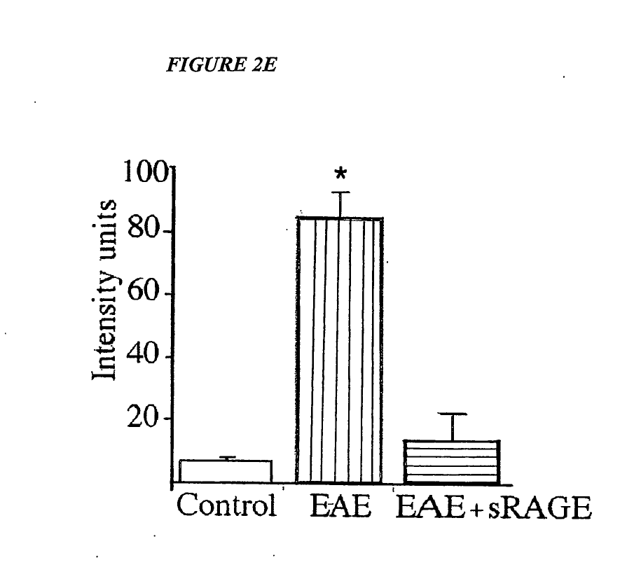

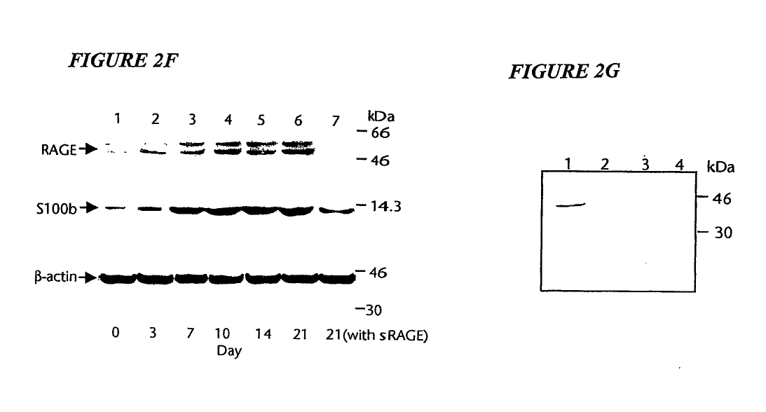

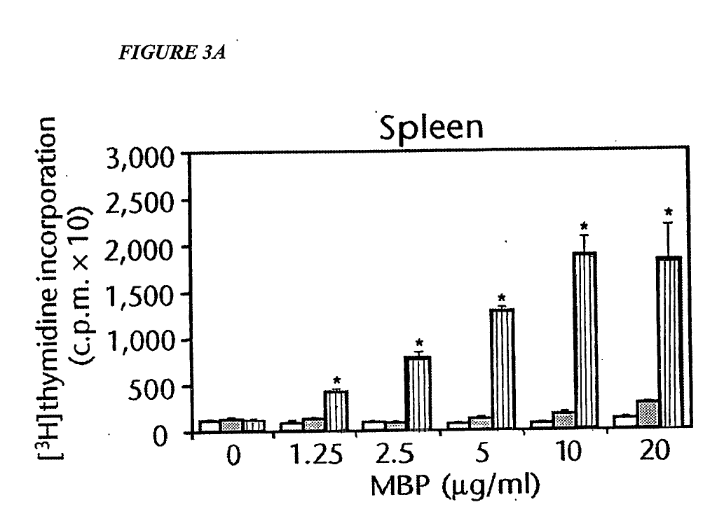

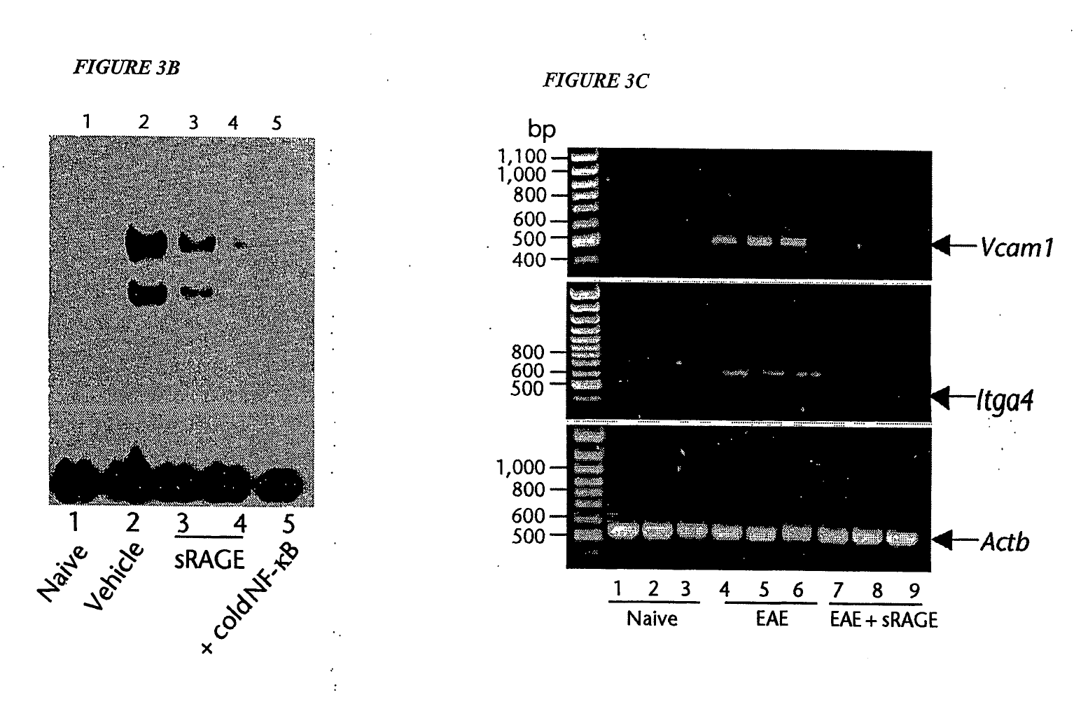

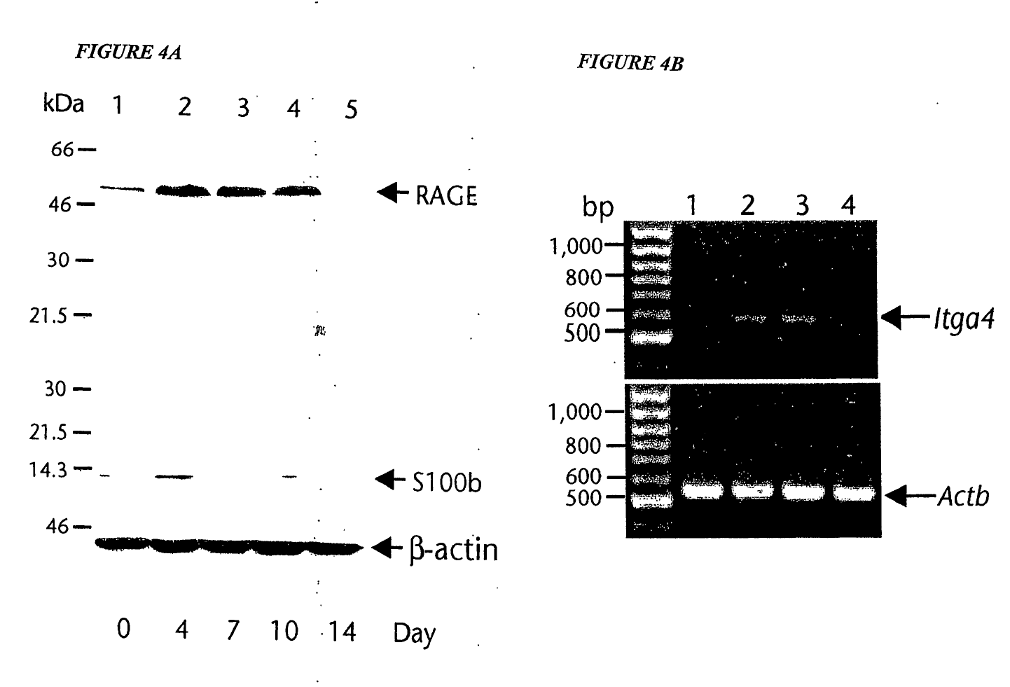

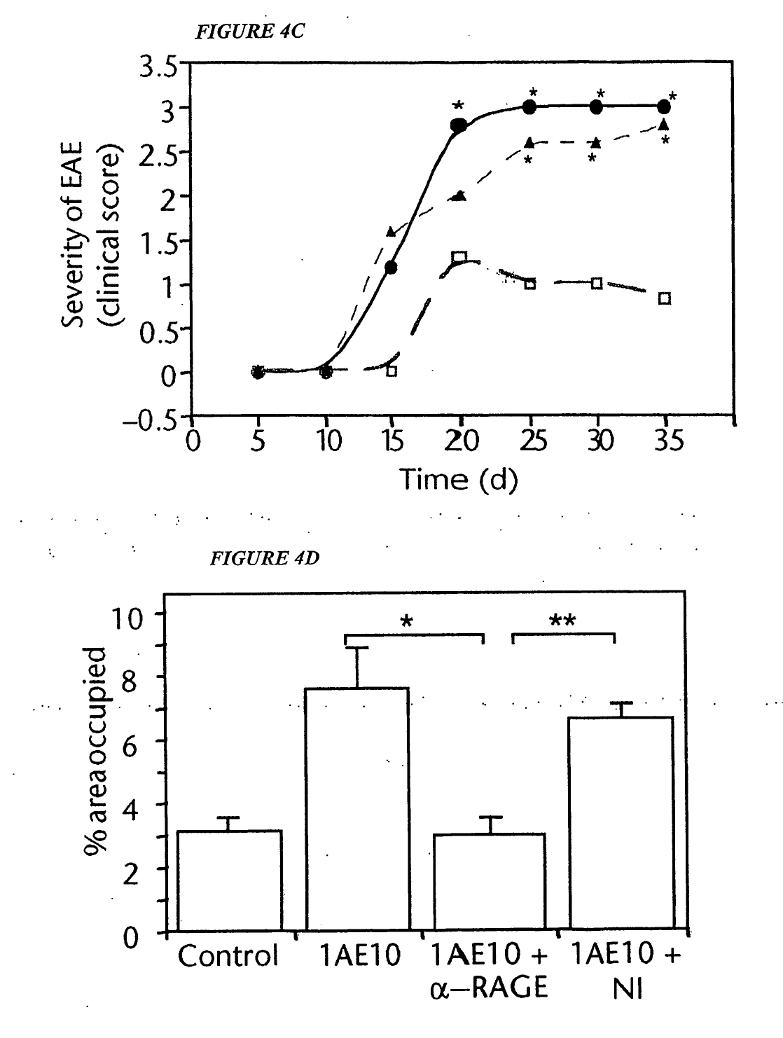

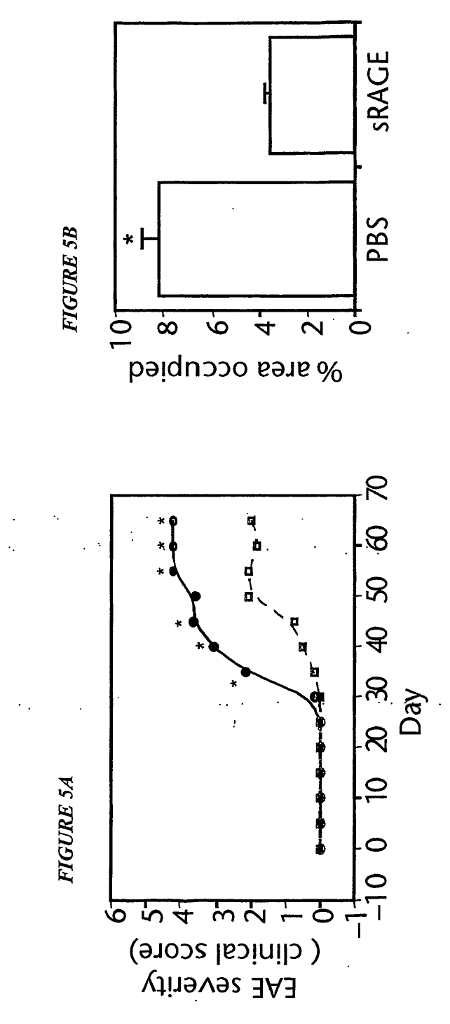

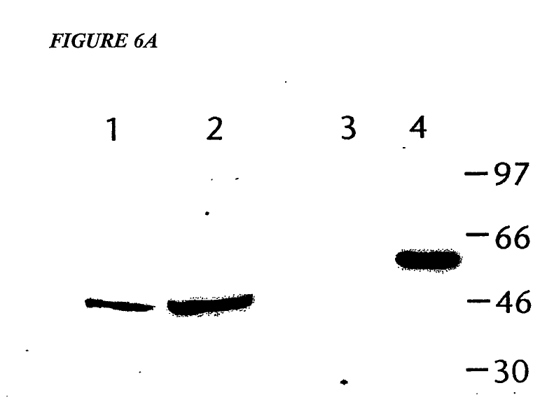

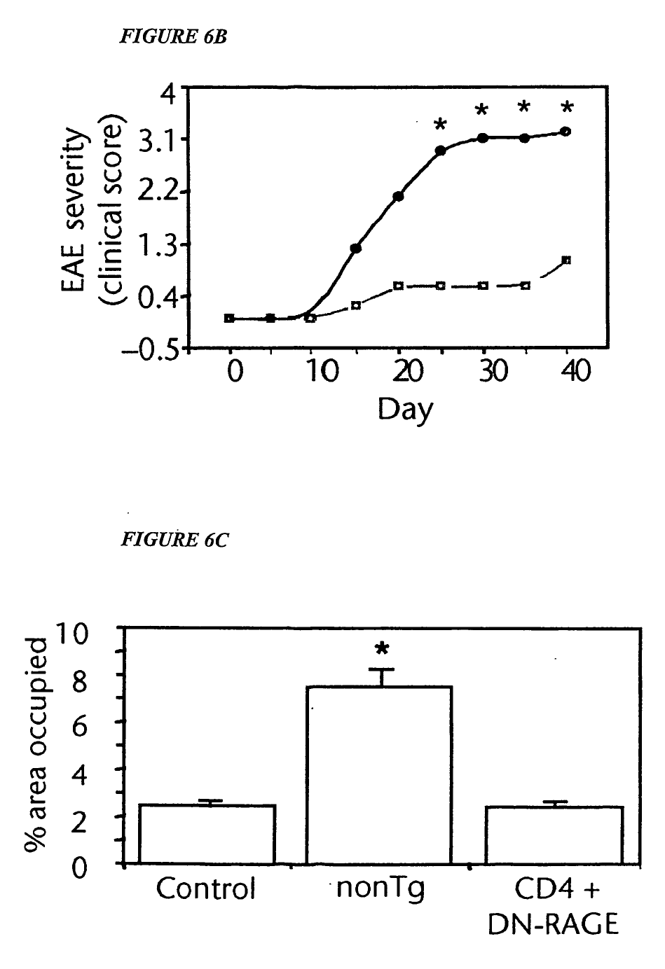

Affected spinal cord from a patient with MS showed inflammatory infiltrates comprised predominately of mononuclear cells (FIG. 1A1), compared with normal spinal cord (FIGS. 1A2; A3 shows image analysis of similar sections comparing area occupied by nuclei, reflecting infiltrating inflammatory cells). RAGE immunoreactivity was increased in the patient sample (FIG. 1B1; versus the control, FIG. 1B2), and was especially evident in mononuclear phagocytes (FIGS. 1D1,2) and CD4+ T-cells (FIGS. 1D3,4), based on colocalization with a macrophage marker (FIGS. 1D1,2) and CD4 (FIGS. 1D 3, 4), respectively. The receptor was also expressed at increased levels in neurons (FIG. 1B1). S100/calgranulin ligands of RAGE were also present at sites of inflammation. They were observed in infiltrating mononuclear cells (FIG. 1C1), as well as neurons (FIG. 1C2). In contrast, levels of S100/calgranulins were much lower in control spinal cord (FIG. 1C3). Immunohistochemical images in FIG. 1 are representative of the analysis of five patients with MS and three age-matched controls. Image analysis, based on data from all of these patients, confirmed an increase in the area of spinal cord occupied by RAGE-positive cells, as well as those bearing S100/calgranulins, in patients compared with controls (FIGS. 1B3, C4).

EAE has proven useful for analysis of pathogenic mechanisms underlying MS (4, 12, 14, 15). Although normal mouse spinal cord showed only low levels of RAGE, spinal cord tissue from animals induced to develop EAE at the time of florid symptomatology demonstrated expression of the receptor. RAGE-positive cells (FIGS. 1E1&3) were evident at the site of inflammatory lesions, and coincided, largely, with MPs (FIG. 1E2) and CD4+ T-cells (FIG. 1E4). Similarly, as was the case with MS patients, S100/calgranulin antigen was increased in spinal cord from mice with EAE (not shown). These data indicate that RAGE and its S100/calgranulin ligands were abundant in the spinal cord of patients with multiple sclerosis and mice undergoing EAE.

II. Blockade of RAGE Protects Animals from EAE: MBP Model.

Expression of RAGE by T-cells and MPs infiltrating the spinal cord, as well as the presence of S100 proteins in tissue from patients with MS and murine EAE, suggested a possible contribution of RAGE-ligand interaction to the pathogenesis of immune/inflammatory demyelinating disease. This issue was first studied using the 1-9NAc MBP (acylated N-terminal peptide comprising nine residues from MBP)-induced model of EAE, and soluble RAGE (sRAGE) to intercept the interaction of ligands with the receptor. Soluble RAGE is a recombinant, truncated form of the receptor spanning the extracellular domain which serves as a decoy binding RAGE ligands and preventing their interaction with cell surface receptor (16). Since low (picomolar) levels of endogenous sRAGE are present in normal plasma, administration of murine sRAGE (50 μg/day) to mice, resulting in micromolar levels in plasma, does not incite an immune response (16). Mice received sRAGE (50 μg/animal) daily by intraperitoneal (IP) injection starting from the day of immunization with 1-9NAc MBP. Although animals immunized with 1-9NAc MBP alone developed prominent symptoms of EAE, administration of sRAGE had a strong protective effect (FIG. 2A). The time at which symptoms were first manifest was delayed, ≈25-30 days versus ≈15-20 days, following the initial immunization, comparing sRAGE and control groups, respectively. Most notably, the severity of symptoms was decreased by sRAGE treatment; average clinical scores at the time of peak symptoms were ≈1.5 versus 4.3, in the sRAGE and control groups, respectively (p<0.001). The protective effect of soluble receptor was dose-dependent over a range of sRAGE concentrations from 2-50 μg/animal (FIG. 2B). Histologic analysis of spinal cord from EAE-induced animals treated with sRAGE, at a time corresponding to peak symptomatic disease in vehicle-treated mice (FIG. 2C2), demonstrated a paucity of inflammatory cells (FIG. 2C3). The latter was comparable to what was observed in spinal cord from unmanipulated control mice (FIG. 2C1). Cellular infiltrates in the different groups were semiquantitated by determining the area occupied by nuclei in H&E-stained sections; there was a significant increase in the nuclear area in EAE-induced animals and this was diminished by treatment with sRAGE (FIG. 2C4).

Mechanisms underlying the effect of sRAGE on symptomatic EAE were assessed at several levels. Based on the known upregulation of RAGE by its ligands (17, 18), it was predicted that administration of sRAGE would prevent accumulation of S100/calgranulins in the tissue and, subsequently, would decrease expression of the receptor. Western blotting showed an increase in immunoreactive full-length RAGE (two closely spaced bands with Mr ≈50-55 kDa) in spinal cord following induction of EAE (time of peak symptoms; FIG. 2D, lanes 4-5), compared with naive controls (lanes 1-3). In contrast, EAE-induced animals treated with sRAGE displayed low levels of spinal cord RAGE at the same time (lanes 6-8). In addition, induction of EAE caused upregulation of S100b in the spinal cord (FIG. 2D, lanes 4-5). Levels of spinal cord S100b antigen in EAE-induced animals were also decreased by treatment with sRAGE (lanes 6-8). Evidence for the in vivo interaction of sRAGE with S100b was obtained by immunoprecipitation/immunoblotting of splenic extracts (FIG. 2E). Spleens were harvested from mice induced to develop EAE and treated with sRAGE, protein extracts were immunoprecipitated with anti-S100b IgG, followed by immunoblotting with antibody to RAGE. Using this protocol, immunoreactive sRAGE antigen (Mr ≈45 kDa) was only identified in EAE-induced mice treated with sRAGE (FIG. 2E, lane 1), not in EAE-induced mice treated with vehicle (lane 2) or naive controls (lane 3). Similarly, when the immunoprecipitation step employed nonimmune IgG, in place of anti-S100b IgG, samples from EAE-induced mice treated with sRAGE did not demonstrate the RAGE-immunoreactive band on subsequent Western blotting with anti-RAGE IgG (lane 4). These data indicated that sRAGE treatment broke the cycle of RAGE-ligand interaction, at least in part by clearing S100/calgranulins from the tissue and down-regulating endogenous RAGE and S100/calgranulins at the site of the immune/inflammatory response.

The pronounced protective effect of sRAGE treatment on EAE leads to the consideration of two possibilities underlying its properties: inhibition of the generation of MBP-reactive cells, especially CD4+ T-cells expressing Th1 cytokines, or preventing CNS infiltration by MBP-specific T-cells. Lymph node and splenic cells were harvested from mice induced to develop EAE between days 15-21, a time when they exhibited strong symptomatology (FIG. 2A) and CNS infiltration by immune/inflammatory cells (FIG. 2C). When splenocytes were in vitro stimulated with 1-9NAc MBP, T-cell proliferation, assayed by 3H-thymidine incorporation, was greater in sRAGE-treated EAE-induced animals than in vehicle-treated animals induced to develop EAE (FIG. 3A1). Similar results, though of lower magnitude, were observed with lymph node cells from sRAGE-treated EAE-induced animals (FIG. 3A2). Furthermore, the Th1/Th2 cytokine profile of lymph node cells from sRAGE treated mice revealed an increase, of at least 2-fold, in cells expressing Th1 (CD4+ g-IFN+ cells) and Th2 (CD4+ IL-5+cells) cytokines (not shown). These data suggested the possibility that either sRAGE enhanced activation of the immune system and/or it retarded migration of potentially encephalitogenic 1-9Nac MBP-reactive T-cells from the periphery into the CNS (thereby promoting their accumulation in peripheral lymph nodes and spleen).

To explore these possibilities, it was first considered whether sRAGE treatment enhanced lymphocyte proliferation or Th1 cell development of encephalitogenic 1-9NAc MBP reactive cells upon antigen stimulation. T-cell proliferation and Th phenotypes of lymph node and splenic cells from mice seven days after in vivo 1-9NAc MBP immunization in the presence/absence of sRAGE were compared. In three separate experiments, there were no significant differences in T-cell proliferation and Th1/Th2 phenotype due to sRAGE treatment (not shown). These data pointed to an effect of sRAGE on migration of potentially encephalitogenic T-cells into the CNS. Of course, other mechanisms might also be operative. For example, T-cells from vehicle-treated EAE-induced mice at peak inflammation might proliferate poorly to MBP due to antigen-induced downregulaton of T-cell receptors, activation-induced cell death and/or other mechanisms. Suppressed inflammation in sRAGE-treated mice might, thus, enhance T-cell proliferation in this setting. However, based on the data described below, effect of sRAGE on the migration of MBP-specific T-cells into the CNS was favored.

Based on these observations, it was reasoned that interception of RAGE-ligand interaction was principally impacting on later events during evolution of the immune/inflammatory response, potentially expression of proinflammatory mediators and influx of inflammatory cells into the CNS. Nuclear translocation of the transcription factor NF-kB is associated with the pathogenesis of the inflammatory response, including in the setting of EAE (19-22). Nuclear extracts prepared from spinal cords of mice induced to develop EAE and harvested at the time of peak symptoms (day 17-21) displayed a strong gel shift band with 32P-labelled consensus probe for NF-kB in electrophoretic mobility shift assays (FIG. 3B, lane 2). Appearance of the latter gel shift band was blocked by inclusion of excess unlabelled NF-kB probe in the incubation mixture with spinal cord nuclear extract (lane 5). Nuclear extracts from animals induced to develop EAE treated with sRAGE (same time point as above, days 17-21) displayed variable, though consistently reduced NF-kB DNA binding activity (FIG. 3B, lanes 3-4). These data were consistent with suppressed cellular activation, probably due to decreased influx of immune/inflammatory cells, in the spinal cord of EAE-induced mice treated with sRAGE.

It was hypothesized that decreased activation of NF-kB in spinal cord extracts from EAE-induced mice treated with sRAGE was likely to be parallelled by diminished transcripts for chemokines/chemokine receptors. Increased expression of the latter has been associated with EAE (23-27). Ribonuclease protection assays for several chemokines demonstrated induction of transcripts for RANTES, MIP-1β, MIP-1a, MIP-2, JE/MCP-1 and TCA-3 (each achieved statistical significance) in spinal cord extracts from EAE-induced mice (time of peak symptoms; FIG. 3C, lanes 4-6) compared with untreated controls (lanes 1-3). Similarly, there was a prominent increase in spinal cord mRNA for chemokine receptors (CCR1, CCR2, CCR5, CXCR2, CXCR4) (FIGS. 3D1-2, lanes 4-6). In each case, animals induced to develop EAE treated with sRAGE did not show an increase in chemokine or chemokine receptor mRNA (FIG. 3C, D1-2, lanes 7-9). Expression of Vascular Cell Adhesion Molecule-1 (VCAM-1) and its counter-receptor, Very Late Activation antigen (VLA)-428, has been associated with EAE and the encephalitogenic potential of T-cell lines (13, 29-31). RT-PCR analysis of spinal cord for VCAM-1 transcripts demonstrated their presence in EAE-induced (day 15; FIG. 3E, lanes 4-6) animals, whereas EAE-induced animals treated with sRAGE (day 15; lanes 7-9) and naive controls (lanes 1-3) had undetectable VCAM-1 mRNA. Levels of VLA-4 mRNA followed a similar pattern (FIG. 3E). Expression/activity of matrix metalloproteinases (MMPs) has been linked to invasivity of encephalitogenic T-cells (31), and increased MMP 9 activity was observed in EAE-induced animals based on zymography (FIG. 3F, lanes 4-6; densitometric analysis of data is shown below the zymogram). Such MMP 9 activity remained at low levels in EAE-induced animals treated with sRAGE (F, lanes 7-9).

III. Induction of EAE by Encephalitogenic T-Cells is Prevented by Inhibition of RAGE.

Although inhibition of RAGE did not appear to impact on initial activation of 1-9NAc MBP-specific lymphocytes, subsequent cellular events (i.e., CNS infiltration) were suppressed based on reduced infiltration and induction of inflammation in the CNS. To more directly analyze the effect of RAGE blockade on encephalitogenic T-cells, an encephalitogenic CD4+ Th1 T-cell clone (1AE10) (32) was used. Two approaches were utilized in these studies, assessment of the effect of RAGE inhibition on the activation of 1AE10 cells in vitro, and determination of the impact of anti-RAGE F(ab′)₂ on EAE induced by adoptive transfer of activated 1AE10 cells in vivo.

1AE10 cells were activated in the presence of 1-9NAc MPB and expression of RAGE and S100/calgranulins was assessed. Immunoblotting showed RAGE expression to be increased by ≈3-fold comparing day 0 with day 4 after stimulation (FIG. 4A). Similarly, S100b antigen increased during 1AE10 cell stimulation (FIG. 4A). In view of the presence of receptor and ligand in activated 1AE10 cells, it was assessed whether blockade of RAGE, achieved with anti-RAGE F(ab′)₂, would modulate properties of 1AE10 cells. Blockade of RAGE with antibody fragments also prevented expression of VLA-4 transcripts by activated 1AE10 cells (FIG. 4B).

These results were promising, in that RAGE inhibition appeared to suppress critical aspects of 1AE10 activation relevant to induction of EAE. However, the likely presence of activated encephalitogenic T-cells in patients with MS indicated the importance of determining whether RAGE blockade could also inhibit development of disease following adoptive transfer of fully activated 1AE10 cells. To address this issue, B10.PL mice were adoptively transferred with activated 1AE10 cells. Animals received either anti-RAGE F(ab′)₂ or nonimmune F(ab′)₂ for 17 days starting at the time of adoptive transfer of 1AE10 cells (FIG. 4C). Symptoms of EAE were observed about 15 days after adoptive transfer of activated 1AE10 cells in animals receiving nonimmune F(ab′)₂, and symptoms progressed rapidly to level 3. These observations contrasted with the strong protective effect observed in animals receiving activated 1AE10 cells and anti-RAGE F(ab′)₂. In the latter case, symptoms only achieved level 0.75-1.2 even after 35 days of observation. Consistent with these results, immunohistologic analysis of spinal cord tissue from these animals showed prominent inflammatory infiltrates in the symptomatic mice treated with nonimmune F(ab′)₂ (FIG. 4D4), whereas there were few inflammatory cells in spinal cord tissue harvested from mice receiving anti-RAGE F(ab′)₂ (FIG. 4D3). Image analysis of area occupied by nuclei per microscopic field revealed increased numbers in EAE-induced mice receiving nonimmune F(ab′)₂ compared with anti-RAGE F(ab′)₂ (p<0.05; panel 4D5). The differences in these counts predominately reflect the infiltrating mononuclear cell population.

The data thus far suggested the possibility that blockade of RAGE might prevent entry of encephalitogenic T-cells into the CNS. To examine this issue directly, activated 1AE10 cells were fluorescently labelled and adoptively transferred into irradiated B10.PL mice in the presence of nonimmune F(ab′)₂ or anti-RAGE F(ab′)₂ (FIG. 4E). Representative microscopic fields from an animal in each group (N=3) are shown (FIGS. 4E1-3), and the increase in fluorescently-labelled cellular profiles was evident in the sample from mice receiving PBS or nonimmune F(ab′)₂ (panels 1&3, respectively) compared with anti-RAGE F(ab′)₂ (FIG. 4E2). Data from animals on day 3 was compared semiquantitatively by counting fluorescently-labelled cells per microscopic field (3-5 fields per animal and 3 animals/per group were studied) (FIG. 4E4). Decreased entry of fluorescent 1AE10 cells into the CNS was a selective effect of anti-RAGE F(ab′)₂, as analysis of lymph node and spleen showed a comparable number of fluorescent cells in each of the groups (nonimmune F(ab′)₂ and anti-RAGE F(ab′)₂) (not shown). These data lead to the assessment of entry of encephalitogenic T-cells into the spinal cord prior to day 3 using 51Cr-labelled activated 1AE10 cells (resulting in a higher specific activity of radiolabelling). In contrast to vehicle-treated controls and animals receiving nonimmune F(ab′)₂, in the presence of anti-RAGE F(ab′)₂, there was strong suppression of 51Cr-radioactivity associated with the spinal cord following adoptive transfer of the radiolabelled 1AE10 cells after 24 hrs (FIG. 4F1). Consistent with a possible mechanism whereby inhibition of RAGE traps encephalitogenic T-cells within the intravascular space (i.e., preventing their migration to the CNS), there was an increase in radioactivity associated with spleen following adoptive transfer of 51Cr-labelled 1AE10 cells along with anti-RAGE F(ab′)₂, compared with nonimmune F(ab′)₂ (FIG. 4F2). Similarly, the liver an increase in 51Cr-associated radioactivity between animals treated with anti-RAG F(ab′)₂ and nonimmune F(ab′)₂ (FIG. 4F3).

Decreased CNS entry of activated 1AE10 cells in the presence of anti-RAGE F(ab′)₂ suggested a role for RAGE-ligand interaction in cellular migration. 1AE10 cells demonstrated directional migration (i.e., chemotaxis) in the presence of increasing levels of S100b added to the lower compartment of chemotaxis chambers (FIG. 4G1). When S100b was placed in the upper compartment of the chemotaxis chamber, the concentration gradient driving cell migration was distorted, and directional cell migration ceased. In the presence of anti-RAGE F(ab′)₂, migration of 1AE10 cells was blocked in a dose-dependent manner, though nonimmune F(ab′)₂ was without effect (FIG. 4G2). Migration of 1AE10 cells to formyl-methionyl-leucinyl-phenylalanine (FMLP) was not affected by RAGE blockade (FIG. 4G3).

In view of the close relationship between cellular invasion and expression of matrix metalloproteinases (MMPs) in the context of EAE (31, 33, 34), the effect of RAGE-dependent stimulation of 1AE10 cells on the activity of MMP 2 (FIG. 4H) was examined. Zymography showed that MMP 2 was increased in activated 1AE10 cells (MMP 9 activity was not detectable in these cells). Addition of anti-RAGE F(ab′)₂, but not nonimmune F(ab′)₂, had a strong inhibitory effect on expression of MMP 2 activity, presumably due to its ability to block the interaction of endogenously generated S100 proteins with cell surface RAGE.

IV. Inhibition of RAGE on CD4+T-cells mediates the protective effect of RAGE blockade.

The presence of RAGE on encephalitogenic CD4+ T-cells and the protective effect of anti-RAGE F(ab′)₂, after administration of activated 1AE10 cells, was consistent with the concept that RAGE on CD4+ cells might be critical for EAE, especially for their infiltration of the CNS. To more directly assess if RAGE blockade impacted on pathogenic properties of encephalitogenic T-cells, experiments were performed with MBP-specific T-cell receptor (TCR) transgenic mice (these mice had transgenes encoding genomic clones of the TCR-a and -β chains obtained from an MBP-specific encephalitogenic CD4+ T-cell clone) (13,35) crossed with mice deficient in endogenous TCR-a and -β chains (T/a-β-) (36). The latter mice have been shown to have MBP-specific TCR expression on virtually all CD4+ T-cells, and all animals develop EAE spontaneously, analogous to transgenic mice bearing the same MBP-specific TCR in the RAG-1 knockout background (37). Compared with T/a-β- animals treated with vehicle, those receiving sRAGE demonstrated a significant protective effect at the level of symptomatic evaluation (FIG. 5A). In addition, histologic studies of the spinal cord demonstrated decreased immune/inflammatory infiltrating cells in sRAGE treated T/a-β- mice (FIGS. 5B; B3 shows image analysis of sections similar to B1-2).

In order to be certain that blockade of RAGE was directly affecting functional properties of CD4+ T-cells, transgenic mice were made expressing a dominant-negative (DN) form of human RAGE under control of the CD4 promoter/enhancer (38,39). DN-RAGE is a truncated form of the receptor devoid of the cytosolic tail, but with intact transmembrane spanning and extracellular domains (6). Previous in vitro studies with transformed murine microglial cells demonstrated that introduction of DN-RAGE prevented RAGE-mediated cellular activation by S100b, even in the presence of endogenous wild-type receptor (6). PCR analysis of a representative litter demonstrates the presence of the CD4-DN-RAGE transgene in positives (a total of four independent founders were identified) and these mice are termed Tg CD4-DN-RAGE; FIG. 6A). CD4+ T-cells isolated from spleens of Tg CD4-DN-RAGE mice subjected to immunoblotting showed a RAGE-immunoreactive band with Mr ≈45 kDa (FIG. 6B, lanes 1-2). The latter would be expected for a truncated form of RAGE lacking the cytosolic tail (the latter comprised of 43 amino acids), i.e., DN-RAGE. In contrast, CD4+ T-cells from Tg mice overexpressing wild-type RAGE (Tg CD4-wtRAGE) displayed a more slowly migrating band with Mr ≈50-55 kDa (lane 4). Similar experiments on CD8+ T-cells, B cells and MPs did not demonstrate expression of the DN-RAGE transgene in Tg CD4-DN-RAGE mice (data not shown).

Functional studies were performed on CD4+ T-cells isolated from spleens of Tg CD4-DN-RAGE mice. Cell migration was assessed by addition of S100b to the lower compartment of chemotaxis chambers; whereas CD4+ T-cells from Tg CD4-DN-RAGE mice did not display cell migration to S100b, CD4+ T-cells from Tg CD4-wtRAGE animals showed a robust response to S100b (1 & 5 μg/ml; FIG. 6C). Consistent with the specificity of RAGE-ligand interactions, although CD4+ T-cells isolated from Tg CD4-DN-RAGE mice showed suppressed migration to S100b, chemotaxis induced by FMLP was intact (not shown). Taken together, these data indicate that the DN-RAGE transgene was selectively expressed in CD4+ T cells, and that it failed to support RAGE-dependent cellular migration.

Tg CD4-DN-RAGE mice and nonTg littermate controls in the B10.PL background were induced to develop EAE using 1-9NAc MBP as in the studies described above (FIG. 2). NonTg littermates demonstrated symptomatic EAE within 12-15 days and symptoms reached a plateau by 30 days (average score 3.2 at this time; FIG. 6D). In contrast, Tg CD4-DN-RAGE mice who underwent the same regimen were virtually asymptomatic (mean scores of 0.6-1.0; FIG. 6D). At the time that nonTg littermates were suffering from symptomatic EAE, animals from both groups were sacrificed so that spinal cord tissue could be analyzed histologically (FIG. 6E). There were prominent inflammatory infiltrates in CNS tissue from nonTg littermates (FIGS. 6E2; E1 shows a section from a naive nonTg mouse). However, there were virtually no cellular infiltrates in Tg CD4-DN-RAGE mice induced to develop EAE (FIG. 6E3; cellular infiltration was not present in naive Tg CD4-DN-RAGE mice, not shown). Area occupied by nuclei in spinal cord sections from five mice in each group confirmed the impression of significantly diminished CNS cellular infiltrates in Tg CD4-DN-RAGE mice compared with controls (FIG. 6E4).

Tg mice were also made with targeted expression of the DN-RAGE transgene in mononuclear phagocytes (MPs) using the macrophage scavenger receptor (MSR) promoter (Tg MSR-DN-RAGE). Characterization of MPs from these animals displayed selective suppression of RAGE-dependent responses, analogous to what was observed in CD4+ T-cells from Tg CD4-DN-RAGE mice (FIG. 6C). Tg MSR-DN-RAGE mice and nonTg littermates were immunized with MBP to induce EAE. The level of symptoms in transgenics was indistinguishable from that observed in nonTg controls (FIG. 6F; data on day 35, last day of the experiment, is shown). Consistent with these observations, infiltration of the CNS by immune/inflammatory cells was identical comparing Tg MSR-DN-RAGE and nonTg littermates (not shown).

Discussion

Biology of the initial stages of EAE, even prior to relapses and establishment of chronic disease, involves multiple steps. Mechanistically, these early events can be divided into two general phases, initiation/establishment of autoimmunity and later processes associated with the evolving immune/inflammatory response (2, 40). The key event underlying initiation is induction of autoimmunity to components of the myelin sheath, whereas later events involve a series of interlocking steps eventuating in destructive inflammation in the CNS. Approaches that address the autoimmunity facet of EAE/MS include administration of peptide-based drugs that interfere with presentation of myelin fragments by the MHC (41, 42), as well as agents targeting costimulatory molecules (43-46). Progression of the nascent autoimmune response involves further development of reactive T-cells with encephalitogenic potential, and their ultimate entry into the CNS (4, 47). Once present in the CNS, recruitment of additional immune/inflammatory cells is critical for establishment of disease (48, 49). This multistep process (4, 47) includes a range of cytokines/lymphokines (TNF-a, g-interferon etc) (4, 47), chemokines/chemokine receptors (23-27), cell adhesion molecules (CD44, VCAM-1, etc) (13, 30, 5.0, 51), and MMPs (31, 33, 34). In addition, glutamate released by immune/inflammatory cells, which potentially exerts cytotoxic effects on receptor-bearing neurons and oligodendrocytes (52, 53), has also been implicated as a factor amplifying the local inflammatory response.

Blockade of RAGE appears to inhibit events in the phase of EAE after the initial activation of T-cells with MBP; i.e., the evolving immune/inflammatory response. Even when an MBP-specific CD4+ Th1 T-cell clone (1AE10) was stimulated with MBP in vitro, blockade of RAGE diminished its encephalitogenic potential in vivo. Based on detection of sRAGE-S100 complexes in sRAGE-treated animals undergoing MBP-induced EAE, as well as the presence of S100/calgranulins and RAGE antigens in an overlapping distribution in MS/EAE-affected spinal cord, it is reasonable to speculate that S100/calgranulin ligands of the receptor are involved in RAGE-mediated inflammatory events in this setting. Furthermore, the presence of RAGE and S100/calgranulins in both immune/inflammatory cells and neurons indicates that consequences of RAGE-ligand interaction for neurons could be direct, as well as indirect, the latter via inflammatory effector cells. In this context, engagement of RAGE on neuronal-like cell lines causes cell stress (activation of NF-kB, expression of chemokines etc) followed, at later times, by cell death (54-56). Thus, analogous to the cytoprotective effects of glutamate receptor antagonists, inhibition of RAGE may diminish neuronal stress at a late point in the inflammatory cascade.

A common theme joining our observations on the effects of RAGE blockade in different experimental models of EAE is that RAGE participates in the activation of MBP-reactive, CD4+ T-cells thereby facilitating their migration into the CNS. In contrast, MBP-immunized mice treated with sRAGE did not display suppression of the initial activation of T-cells to antigen or in vivo differentiation to Th1 versus Th2 cells. Consistent with the hypothesis that RAGE impacts on properties of CD4+ T-cells after their initial exposure to antigen, activation of an encephalitogenic CD4+ Th1 T-cell clone (1AE10) with MBP was clearly influenced by the presence of anti-RAGE F(ab′)₂, based on inhibition of expression of VLA-4. At a still later point in the inflammatory cascade, when fully activated 1AE10 cells were adoptively transferred into B10.PL mice, induction of EAE was also prevented by RAGE blockade. Furthermore, inhibition of EAE occurred in parallel with decreased entry of 1AE10 cells into the spinal cord. It is speculated that key mechanisms underlying the effect of RAGE inhibition on EAE in these settings is modulation of the expression of chemokines/chemokine receptors and cell adhesion molecule receptors/counter-receptors (i.e., VLA-4/VCAM-1). Although further studies will be required to address this hypothesis, the results of the in vitro experiments are consistent with this general concept based on RAGE-ligand-dependent induction of 1AE10 cell chemotaxis and VLA-4 expression. These data emphasize the likelihood that the RAGE-ligand axis is operative, at least in part, at a late stage in the inflammatory process. That RAGE-dependent effects in this context were likely to occur, at least in part, at the level of CD4+ T-cells, rather than CD8+ T-cells or MPs, was suggested by our observations in T/a-β-animals and transgenic mice with targeted expression of dominant-negative RAGE. Effective inhibition of EAE in T/a-β- treated with sRAGE and in Tg CD4-DN-RAGE mice, contrasted with lack of impact on EAE symptomatology in mice bearing the DN-RAGE transgene selectively expressed in MPs. Taken together, these data are consistent with the focus on the role of RAGE in the biology of CD4+ T-cells for analysis of mechanisms underlying EAE/MS.

Previous studies have highlighted contributions of chemokines (23-27), VCAM-1/VLA-4 (13, 30) and MMPs (31, 33, 34), as well as other inflammation-associated cofactors (4, 47), in the pathogenesis of EAE following initial activation of lymphocytes to myelin sheath-associated proteins. At the mRNA level, inhibition of RAGE caused strong reductions in expression of transcripts for a range of chemokines, as well as VLA-4, VCAM-1, and MMPs. Although it is difficult to be certain which of these events may prove most important for the apparent affect of RAGE on CD4+ T-cell activation/migration into the CNS, it is clear that RAGE-ligand interaction impacts on MBP-primed CD4+ T-cells. It is proposed that blockade of RAGE may ultimately represent a novel approach for treating MS, potentially effective after lymphocytes reactive with myelin sheath components are present and have undergone early stages of activation. Although relapsing-remitting models have not yet been analyzed, the spontaneous model better mimics chronic progressive MS than any other aspect of MS.

### REFERENCES

1. Bauer, J., Rauschka, H. & Lassmann, H. Inflammation in the nervous system: the human perspective. Glia 36, 235-243 (2001).
2. Hohlfeld, R. & Wekerle, H. Immunological update on multiple sclerosis. Curr Opin Neurol 14, 299-304 (2001).
3. Zamvil, S., et al. T cell clones specific for myelin basic protein induce chronic relapsing paralysis and demyelination. Nature 317, 355-358, 1985.
4. Owens, T., Wekerle, H. & Antel, J. Genetic models for CNS inflammation. Nat Med 7, 161-166 (2001).
5. Schmidt, A-M., Yan, S-D., Yan, S—F. & Stern, D. M. The multiligand receptor RAGE as a progression factor amplifying immune and inflammatory responses. J Clin Invest 108, 949-955 (2001).
6. Hofmann, M., et al. RAGE mediates a novel proinflammatory axis: the cell surface receptor for S100/calgranulin polypeptides. Cell 97, 889-901 (1999).
7. Taguchi, A., et al. Blockade of RAGE/amphoterin suppresses tumor growth and metastases. Nature 405, 354-360 (2000).
8. Rauvala, H., et al. The adhesive and neurite-promoting molecule p30: analysis of the amino terminal sequence and production of antipeptide antibodies that detect p30 at the surface of neuroblastoma cells and of brain neurons. J Cell Biol 107, 2293-2305 (1987).
9. Hori, O., et al. RAGE is a cellular binding site for amphoterin: mediation of neurite outgrowth and co-expression of RAGE and amphoterin in the developing nervous system. J Biol Chem 270, 25752-25761 (1995).
10. Schafer, B. & Heizmann, C. The S100 family of EF-hand calcium-binding proteins: functions and pathology. TIBS 21, 134-140 (1996).
11. Wong F., Dittel, B. & Janeway, C. Transgenes and knockout mutations in animal models of type 1 diabetes and multiple sclerosis. Immunol Rev 169, 93-106 (1999).
12. Swanborg, R. EAE in rodents as a model for human demyelinating disease. Clin Immunol 77, 4-13 (1995).
13. Baron, J., Madri, J., Ruddle, N., Hashim, G. & Janeway, C. Surface expression of a4-integrin by CD4 T cells is required for their entry into brain parenchyma. J Exp Med 177, 57-68 (1993).
14. Fazekas, G. & Tabira, T. What transgenic and knockout models teach us about experimental autoimmune encephalitis. Rev Immunogenet 2, 115-132 (2000).
15. Heber-Katz, E. The relationship between human multiple sclerosis and rodent experimental allergic encephalomyelitis. Ann New York Acad Sci 756, 283-293 (1995).
16. Park, L., et al. Suppression of accelerated diabetic atherosclerosis by sRAGE. Nat Med 4, 1025-1031 (1998).
17. Li, J. & Schmidt, A-M. Characterization and functional analysis of the promoter of RAGE. J Biol Chem 272, 16498-16506 (1997).
18. Li, J., Qu, W. & Schmidt, A-M. Sp1 binding elements in the promoter of RAGE are essential for amphoterin-mediated gene expression in cultured neuroblastoma cells. J Biol Chem 273, 30870-30878 (1998).
19. Baeuerle, P. & Henkel, T. Function and activation of NF-kB in the immune system. Annu Rev Immunol 12, 141-179 (1994).
20. Kaltschmidt, C., et al. Transcription factor NF-kB is activated in microglia during EAE. J Neuroimmunol 55, 99-106 (1994).
21. Pahan K. & Schmid, M. Activation of NF-kB in the spinal cord of EAE. Neurosci Lett 287, 17-20 (2000).
22. Bonetti, B., et al. Activation of NF-kB and c-jun transcription factors in MS lesions. Implications for oligodendrocyte pathology. Am J Pathol 155, 1433-1438 (1999).
23. Nygardas, P., Maatta, J. & Hinkkanen, A. Chemokine expression by CMS resident cells and infiltrating neutrophils during EAE in the Balb/c mouse. Eur J Immunol 30, 1911-1918 (2000).
24. Matejuk, A., Vandenbark, A., Burrows, G., Bebo, B. & Offner, H. Reduced chemokine and chemokine receptor expression in spinal cords of TCR BV82S transgenic mice protected against EAE with BV82S protein. J Immunol 164, 3924-3931 (2000).
25. Karpus, W. & Ransohoff, R. Chemokine regulation of EAE: temporal and spatial expression patterns govern disease pathogenesis. J Immunol 161, 2667-2671 (1998).
26. Fife, B., Huffnagle, G., Kuziel, W. & Karpus W. CC chemokine receptor 2 is critical for induction of EAE. J Exp Med 192, 899-905 (2000).
27. Rottman, J., et al. Leukocyte recruitment during onset of EAE is CCR1 dependent. Eur J Immunol 30, 2372-2377 (2000).
28. Carlos, T., et al. VCAM-1 mediates lymphocyte adherence to cytokine-activated cultured human endothelial cells. Blood 76, 965-970 (1990).
29. Madri, J., Graesser, D. & Haas, T. The roles of adhesion molecules and proteinases in lymphocyte transendothelial migration. Biochem Cell Biol 74, 749-757 (1996).
30. Yednock, T., et al. Prevention of EAE by antibodies against a4β1-integrin. Nature 356, 63-66 (1992).
31. Graesser, D., Mahooti, S. & Madri, J. Distinct roles for MMP-2 and a4-integrin in autoimmune T cell extravasation and residency in brain parenchyma during EAE. J Neuroimmunol 109, 121-131 (2000).
32. Jiang, H., Braunstein, N., Yu, B., Winchester, R. & Chess, L. CD8 T cells control the Th phenotype of MBP-reactive CD4 T cells in EAE mice. Proc Natl Acad Sci (USA) 98, 6301-6306 (2001).
33. Gijbels, K., Galard, R. & Steinman, L. Reversal of EAE with an hydroxamate inhibitor of matrix metalloproteases. J Clin Invest 94, 2177-2182 (1994).
34. Liedtke, W., et al. Effective treatment of models of MS by matrix metalloproteinase inhibitors. Ann Neurol 44, 35-46 (1998).
35. Hafler, D. & Weiner, H. Immunologic mechanisms and therapy in MS. Immunol Rev 144, 75-107 (1995).
36. Olivares-Villagomez, D., Wang, Y. & LaFaille, J. Regulatory CD4+ T cells expressing endogenous T cell receptor chains protect myelin basic protein-specific transgenic mice from spontaneous autoimmune encephalomyelitis. J Exp Med 188, 1883-1894 (1998).
37. Lafaille, J., Nagashima, K., Katsuki, M. & Tonegawa, S. High incidence of spontaneous EAE in immunodeficient anti-MBP TCR transgenic mice. Cell 78, 399-408 (1994).
38. Adlam, M., Duncan, D., Ng, D. & Siu, G. Positive selection induces CD4 promoter and enhancer function. Intl Immunol 9, 877-887 (1997).
39. Dunacan, D., Adlam, M., Siu, G. Asymmetric redundancy in CD4 silencer function. Immunity 4, 301-311 (1996).
40. Liblau, R., et al. Demyelinating diseases: from pathogenesis to repair strategies. Trends in Neurosciences 24, 134-135 (2001).
41. Neuhaus, O., Farina, C., Wekerle, H. & Hohlfield, R. Mechanisms of action of glatiramer acetate in multiple sclerosis. Neurol 56, 702-708 (2001).
42. Farina, C., et al. Treatment of multiple sclerosis with Copaxone: Elispot assay detects IL-4 and g-interferon response in blood cells. Br 124 (pt 4), 705-719 (2001).
43. Howard, L., et al. Mechanisms of immunotherapeutic intervention by anti-CD40L antibody in an animal model of MS. J Clin Invest 103, 281-290 (1999).
44. Laman, J., DeBoer, M. & Hart, B. CD40 in clinical inflammation: from MS to atherosclerosis. Dev Immunol 6, 215-222 (1998).
45. Racke, M., Ratts, R., Arredondo, L., Perrin, P. & Lovett-Racke, A. The role of costimulation in autoimmune demyelination. J Neuroimmunol 107, 205-215 (2000).
46. Girvin, A., et al. A critical role for B7/CD28 costimulation in EAE: a comparative study using costimulatory molecule-deficient mice and monoclonal antibody blockade. J Immunol 164, 136-143 (2000).
47. Wingerchuk, D., Lucchinetti, C. & Noseworthy, J. MS: current pathophysiological concepts. Lab Invest 81, 263-281 (2001).
48. Zamvil, S. & Steinman, L. The T lymphocyte in EAE. Annu Rev Immunol 8, 579-621 (1990).
49. Raine, C. The Dale E. McFarlin Memorial Lecture: the immunology of the MS lesion. Ann Neurol 36, S61-S72 (1994).
50. Brocke, S., Piercy, C., Steinman, L., Weissman, I. & Veroma, T. Antibodies to CD44 and integrin-a4, but not L-selectin, prevent CNS inflammation and EAE by blocking secondary leukocyte recruitment. Proc Natl Acad Sci (USA) 96, 6896-6901 (1999).
51. Kobayashi, Y., et al. Antiboides against LFA-1 and ICAM-1 together suppress the progression of EAE. Cell Immunol 46, 295-303 (1995).
52. Pitt, D., Werner, P. & Raine, C. Glutamate excitotoxicity in a model of MS. Nat Med 6, 67-70 (2000).
53. Smith, T., Groom, T., Zhu, B. & Turski, L. Autoimmune encephalomyelitis ameliorated by AMPA antagonists. Nat Med 6, 62-66 (2000).
54. Sousa, M., Yan, S-D., Stern, D. & Saraiva, M. Interaction of RAGE with transthyretin triggers NF-kB activation. Lab Invest 80, 1101-1110 (2000).
55. Sousa, M., et al. FAP: RAGE-dependent triggering of neuronal inflammatory and apoptotic pathways. J Neurosci 21, 7576-7586 (2001).
56. Huttunen, H., et al. Coregulaton of neurite outgrowth and cell survival by amphoterin and S100 proteins through RAGE activation. J Biol Chem 275, 40096-40105 (2000).
57. Wu, C., Moulton, K., Horval, A., Parik, S. & Glass C. Combinatorial interaction between AP-1 and ets domain proteins contribute to the developmental regulation of the MSR gene. Molec Cell Biol 14, 2129-2139 (1994).
58. Jiang, H., Zhang, S-L. & Pernis, B. Role of CD8+ T cells in murine EAE. Science 256, 1213-1215 (1992).
59. Lafaille, J., et al. MBP-specific Th2 cells cause EAE in immunodeficient hosts rather than protect them from disease. J. Exp. Med. 186, 307-312 (1997).
60. Yan, S-D., et al. Nonenzymatically glycated tau in AD induces neuronal oxidant stress resulting in cytokine gene expression and release of AB. Nature Med 1, 693-699 (1995).

1. A method for treating a subject afflicted with multiple sclerosis comprising administering to the subject a therapeutically effective amount of soluble receptor for advanced glycation endproducts (sRAGE).

2. The method of claim 1, wherein the subject is human.

3. The method of claim 1, wherein the therapeutically effective amount of sRAGE is an amount between about 150 μg sRAGE/kg of subject/day and 15 mg sRAGE/kg of subject/day, or its equivalent.

4. The method of claim 1, wherein the therapeutically effective amount of sRAGE is an amount between about 500 μg sRAGE/kg of subject/day and 5 mg sRAGE/kg of subject/day, or its equivalent.

5. The method of claim 1, wherein the therapeutically effective amount of sRAGE is about 1.5 mg/kg of subject/day, or its equivalent.

6. A method for inhibiting CD4⁺ T-cell migration comprising contacting the CD4⁺ T-cell with soluble receptor for advanced glycation endproducts (sRAGE).

7. The method of claim 6, wherein the CD4⁺ T-cell is a human cell.

8. The method of claim 6, wherein the CD4⁺ T-cell is present in a subject, and the contacting with sRAGE is performed by administering a therapeutic amount of sRAGE to the subject.

9. The method of claim 8, wherein the subject is human.

10. The method of claim 8, wherein the therapeutically effective amount of sRAGE is an amount between about 150 μg sRAGE/kg of subject/day and 15 mg sRAGE/kg of subject/day, or its equivalent.

11. The method of claim 8, wherein the therapeutically effective amount of sRAGE is an amount between about 500 μg sRAGE/kg of subject/day and 5 mg sRAGE/kg of subject/day, or its equivalent.

12. The method of claim 8, wherein the therapeutically effective amount of sRAGE is about 1.5 mg/kg of subject/day, or its equivalent.

13. A method for inhibiting chemokine receptor activation in a subject comprising administering to the subject a therapeutically effective amount of soluble receptor for advanced glycation endproducts (sRAGE).

14. The method of claim 13, wherein the subject is human.

15. The method of claim 13, wherein the chemokine receptor is selected from the group consisting of CCR1, CCR2, CCR5, CXCR2, CXCR4, VCAM-1, VLA-4, MMPS receptor, RANTES receptor, MIP-1β receptor, MIP-1a receptor, MIP-2 receptor, JE/MCP-1 receptor and TCA-3 receptor.

16. The method of claim 13, wherein the therapeutically effective amount of sRAGE is an amount between about 150 μg sRAGE/kg of subject/day and 15 mg sRAGE/kg of subject/day, or its equivalent.

17. The method of claim 13, wherein the therapeutically effective amount of sRAGE is an amount between about 500 μg sRAGE/kg of subject/day and mg sRAGE/kg of subject/day, or its equivalent.

18. The method of claim 13, wherein the therapeutically effective amount of sRAGE is about 1.5 mg/kg of subject/day, or its equivalent.

19-21. (canceled)
